(19) United States
(12) Patent Application Publication
(10) Pub. No.: US 2017/0281075 A1
(43) Pub. Date: Oct. 5, 2017

(54) SENSOR-BASED CONDITION MONITOR

(71) Applicant: Cognitive Health LLC, Boston, MA (US)

(72) Inventor: Bill Younker, Boston, MA (US)

(21) Appl. No.: 15/509,586

(22) PCT Filed: Sep. 11, 2015
(86) PCT No.: PCT/US15/49613

§ 371 Date: Mar. 8, 2017

Related U.S. Application Data
(60) Provisional application No. 62/049,611, filed on Sep. 12, 2014.

(51) Int. Cl. A61B 5/00; A61B 5/0205; A61B 5/0476; A61B 5/11

(52) U.S. Cl. CPC A61B 5/486; A61B 5/7221; A61B 5/6803; A61B 5/1121; A61B 5/02055; A61B 5/4806; A61B 5/0476; A61B 5/0024; A61B 5/681; A61B 5/7405; A61B 5/742; A61B 5/7455; A61B 5/0022; A61B 5/6898; A61B 5/02438

(57) ABSTRACT

A sensor-based system that measures at least one parameter relating to an individual and provides feedback to the individual when any of the measured parameters exceed a threshold value for that parameter. In some instances, the system can be used to monitor an individual recovering from a condition, for example, mild Traumatic Brain Injury. The system can include a wearable sensor that measures parameters, which may relate to the individual's movement, physiological function, and/or environment. The system can include a controller that receives the parameter measurements and determines whether such measurements are acceptable by comparison to a threshold value. The system can also include a feedback device that alerts the individual when a measured parameter is unacceptable, for example, with a visual, audible, and/or haptic cue, as well as the capability to receive input from the user when an otherwise acceptable parameter level is causing discomfort.

### CROSS-REFERENCE TO RELATED APPLICATION

This application claims priority to co-pending U.S. provisional patent application Ser. No. 62/049,611, titled “Wearable Sensor-Based Condition Monitor,” filed on Sep. 12, 2014, the disclosure of which is herein incorporated by reference in its entirety.

### TECHNICAL FIELD

In general, various embodiments of this invention relate to methods and systems for monitoring an individual and, more specifically, to a sensor-based system that measures at least one parameter and provides the individual with feedback when the parameter exceeds a threshold value.

### BACKGROUND

Recovery from many conditions and/or injuries requires a progressive approach in which the recovering individual gradually increases activity level and/or environment exposure until a full recovery is made. One such injury is mild Traumatic Brain Injury (“mTBI”), an example of which is often referred to as a concussion. In some instances in this application, mTBI is referred to using the term concussion. According to the United States Centers for Disease Control and Prevention, approximately two million individuals were diagnosed with mTBI in U.S. hospital emergency departments in 2010. Sports related concussion in the US is estimated to occur between 1.8 and 3.8 million times per year, and is expected to grow due to increased awareness resulting from headline National Football League lawsuits, state legislation, and medical science advancements. Despite the rapid growth in awareness, the standard of care for recovery from mTBI itself lags substantially. As an example, only 2-12% of emergency departments provide written discharge instructions that include recommendations corresponding to current best practices for mTBI recovery. In most cases, instead of proactive management, as is the case with most other injuries, a “wait and see” approach is employed. Even if appropriate instructions are given, they are typically several pages long and require the patient to establish and adhere to complex guidelines, such as maintaining heart rate zones and sensory exposures. Not surprisingly, a patient who has been diagnosed with a brain injury is often unable or unwilling to undertake such cognitively taxing self-monitoring. As a result, patient compliance is poor, which negatively affects patient productivity and patient outcomes, as well as increases medical costs.

Accordingly, there exists a need for improved methods for monitoring and/or assisting individuals recovering from certain conditions and/or injuries, for example, mTBI.

### SUMMARY OF THE INVENTION

In certain embodiments, the present invention relates to a sensor-based system that measures one or more parameters (e.g., those related to movement, physiological function, and/or environment) of an individual and provides feedback when such parameters exceed a threshold value. In some cases, the threshold values can be periodically increased, such that an individual is gradually permitted to experience and/or be exposed to increased parameter levels. One beneficial use of the system is in facilitating the recovery from certain injuries, for example, mTBI. Recovery from conditions such as mTBI typically requires the proactive management of physical and cognitive rest. If individuals push themselves too hard, they can be at an increased risk for a longer recovery. With current approaches, it is difficult to establish, monitor and adhere to appropriate levels of rest. While the brain cannot be physically immobilized like other injuries, low cost sensor technology now makes it possible to measure an individual's physical activity, cognitive activity, sensory exposure, and sleep. By providing real time feedback based on these measurements, individuals can be directed to participate in an appropriate amount of physical and cognitive activity and to increase quality sleep.

In embodiments in which the system of the present invention is used to monitor individuals with mTBI, the system includes at least the following improvements over the prior art. First, the system offers a proactive approach that enables an individualized, progressive return to full activity levels, as opposed to the current approach in which most individuals are treated reactively after symptoms have gone untreated or improperly treated. Experts agree that a progressive return to full health is an effective way to manage recovery from certain conditions, such as mTBI. For example, an individual diagnosed with such a condition should keep physical and cognitive activity and sensory exposure to a minimum level at the outset of the diagnosis. Once the individual is symptom free at the initial activity level, the individual may engage in an increased level of activity until symptom free (or until symptoms have changed an acceptable amount) at that level. This progressive, step-based approach is continued until the individual is symptom free (or until symptoms are acceptable) at pre-injury levels and cleared for normal activity. In alignment with this approach, certain embodiments of the system of the present invention can increase threshold values that govern activity levels automatically, if the patient does not alert the system (e.g., through engaging an interface) that the patient is currently symptomatic or otherwise experiencing discomfort or distress. In other embodiments, the threshold values can be increased manually by the patient and/or a third party (e.g., a caregiver, nurse, athletic trainer, etc.).

In addition, the system can perform the difficult and often neglected tasks of monitoring an individual's recovery parameters and informing the individual when such parameters have exceeded a safe threshold value, while gradually pushing the individual back to normal activity through periodic increase of the threshold values. In the absence of such a system, these difficult tasks may need to be undertaken by a caregiver (e.g., spouse, parent, or guardian) or doctor who generally do not have the time and/or skills for such time-consuming, specialized treatment. Further, because some conditions such as mTBI have no outwardly visible signs, they can be difficult to recognize as an injury for both patients and their family, co-workers, and peers. This lack of visibility often leads to questions about the legitimacy of the injury in social, work, and school settings. In some instances, the sensor-based system of the present invention may provide a visible recovery tool that informs others that the individual wearing the system is recovering from a condition and/or injury.

Another benefit of the present invention is that it can collect data related to certain conditions and transfer such data to a centralized database which can facilitate research, access by the individuals and/or caregivers, archiving of data, etc.

In general, in one aspect, embodiments of the invention feature a wearable, sensor-based system for monitoring an individual. The system may include a sensor adapted to measure a parameter related to at least one of movement, a physiological function, and environment of the individual; a controller adapted to receive sensor measurement and determine whether the sensor measurement is acceptable by comparison to a threshold value; and a feedback device in communication with the controller adapted to alert the individual when the sensor measurement is not acceptable, where the controller is further adapted to periodically increase the threshold value.

In various embodiments, the sensor includes a plurality of different parameter measurement elements. In some instances, the sensor can include a headband adapted to be worn by the individual. The movement parameter can be selected from the group consisting of linear acceleration, rotational acceleration, and angular acceleration of a head of the individual, and combinations thereof The physiological function parameter can be selected from the group consisting of heart rate, body temperature, and amount of sleep. The environment parameter can be selected from the group consisting of ambient light level and ambient noise level. In some instances, the feedback device can include a wearable device separate from the sensor, for example, a wristband. The feedback device may alert the individual with at least one of a visual cue, an audible cue, and a haptic cue. At least one of the feedback device and the sensor may communicate wirelessly with the controller. In some instances, the controller is further adapted to receive input from the individual indicating the presence or absence of discomfort and, in some cases (e.g., if the presence of discomfort is indicated), to decrease the threshold value to a previous level in response to such input. The feedback device may include a mobile telecommunications device (e.g., a smartphone or smartwatch). In some cases, the measured parameter relates to mobile telecommunications device usage. In some embodiments, the controller is further adapted to communicate data to a remote data storage device, which may include a server that hosts the communicated data on a web portal.

In general, in another embodiment, embodiments of the invention feature a method of using a wearable, sensor-based device for monitoring an individual. The method may include the steps of measuring a parameter related to at least one of movement, a physiological function, and environment of the individual; determining whether the measured parameter is acceptable by comparison to a threshold value; alerting the individual when the measured parameter is not acceptable; and periodically increasing the threshold value.

In various embodiments, the step of measuring a parameter includes measuring a plurality of different parameters. The movement parameter can be selected from the group consisting of linear acceleration, rotational acceleration, and angular acceleration of a head of the individual, and combinations thereof The physiological function parameter can be selected from the group consisting of heart rate, body temperature, cognitive activity and amount of sleep. The environment parameter can be selected from the group consisting of ambient light level and ambient noise level. In some instances, the step of alerting the individual includes providing the individual with at least one of a visual cue, and audible cue, and a haptic cue. The method may further include the steps of receiving input from the individual indicating the presence or absence of discomfort and altering (e.g., decreasing) the threshold value upon receipt of the input. The method may further include the step of communicating data to a remote data storage device.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic diagram of a wearable, sensor-based condition monitor system according to one embodiment;

FIG. 2 is a chart showing a set of exemplary acceptable values of some measured parameters at various levels, according to one embodiment;

FIG. 3 is a flow diagram showing transfers of collected data, according to one embodiment; and

FIG. 4 is a flow diagram showing a method for monitoring an individual according to one embodiment.

### DESCRIPTION

Embodiments of the present invention are directed to a sensor-based system that measures at least one parameter related to an individual and provides feedback to the individual when such parameter exceeds (or in some cases, falls below) a threshold value. Although this disclosure primarily describes a system for use in conjunction with the treatment of mTBI, in other embodiments the system can be used to monitor any condition for which a progressive recovery approach is desired. Further, in general, the system can be used in any situation in which a parameter is measured, feedback is provided when the parameter extends beyond a threshold value, and the threshold value is periodically altered, for example, in training applications for use with athletes. Certain embodiments of the system are described in greater detail below with reference to the accompanying drawings.

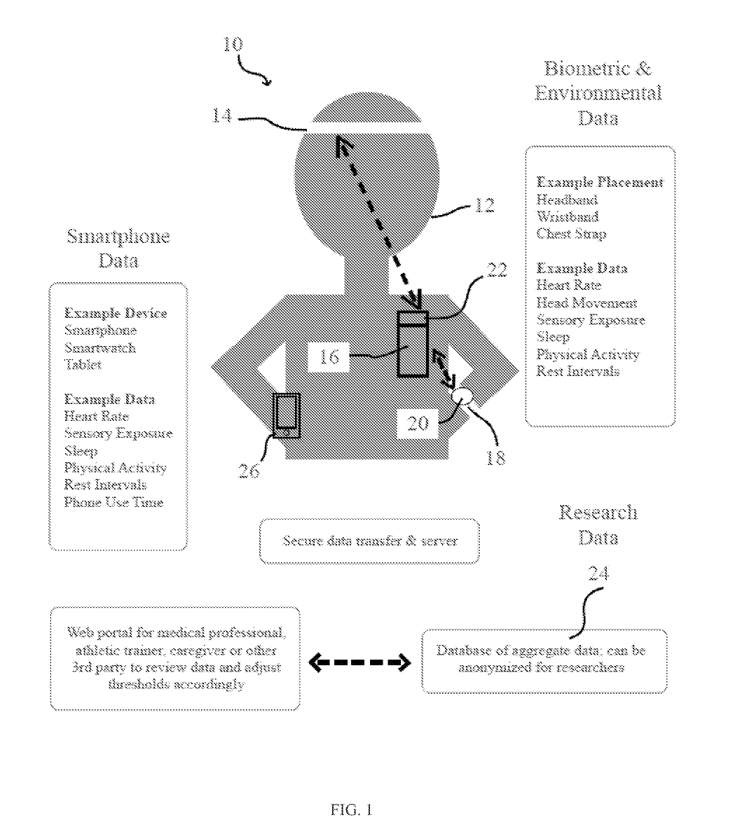

In various embodiments, as depicted for example in FIG. 1, the present invention includes a wearable, sensor-based system 10 for monitoring an individual 12, which in some cases can assist the individual 12 in recovering from an injury (e.g., mTBI). The system 10 may include a sensor 14 adapted to measure various parameters related to the individual 12. In general, the sensor 14 can be placed on the individual's body and/or worn by the individual 12 at a location that allows the sensor 14 to gather appropriate measurements. For example, the sensor 14 can be a headband, or included within a headband, skullcap, or hat, worn by the individual 12. Other examples of the sensor 14 can include a wristband, an armband, a skin patch, or a device that clips to the individual's belt or clothing. In certain embodiments, the sensor 14 can be a mobile device 26 carried by the individual 12. The parameters measured by the sensor 14 can include any measureable item for which the individual 12 requires feedback. In some cases, the parameters can relate to the individual's recovery from an injury such as the individual's movement, physiological functions, and/or environment. More specific examples of parameters that can be measured are described below.

In various embodiments, the system 10 includes a controller 16 adapted to receive a parameter measurement from the sensor 14 and determine whether the measurement is acceptable, for example, by comparison to a threshold value or a range of acceptable values. The threshold value can be a value beyond which the parameter should not extend or, in some cases, fall below, and such a value can be programmed into the controller 16 for each parameter measured by the sensor 14. In some cases, the threshold values can be set based upon appropriate values for an individual recovering from a particular injury.

The system can also include a feedback device 18 in communication with the controller 16 and/or the sensor 14. In situations in which the parameter measurement taken by the sensor 14 is not acceptable by comparison to the threshold value or range, the feedback device 18 can alert the individual 12. In general, the feedback device 18 can be any device capable of drawing the attention of the individual 12. The feedback device 18 can be a wearable item, for example, a headband, wristband, skin patch, device that clips to the individual's belt or clothing, or a device carried by the individual 12. In certain embodiments, the feedback device 18 is the mobile device 26 (e.g., a smartphone, smart watch, or tablet computing device). Thus, the mobile device 26 may be the sensor 14 and/or the feedback device 18. In some such embodiments, alerts can be provided through execution of an application located on the mobile device 26. As described below, in some instances the mobile device 26 can also receive inputs from the monitored individual 12 (e.g., to manually alter threshold levels, or to indicate either a presence or an absence of discomfort). In some cases, the feedback device 18 can be included in the same device as the sensor 14. In other cases, the feedback device 18 can be a separate device from the sensor 14. For example, the sensor 14 can be a headband and the feedback device 18 can be a wristband. The controller 16 can be included in the same device as either the sensor 14 or the feedback device 18, or in some cases can be included in a standalone device. Communication among the sensor 14, the controller 16, and the feedback device 18 may occur through wired or wireless communication.

In general, the feedback device 18 can alert the individual 12 in any way that draws the individual's attention, for example, with a visual, audible, and/or haptic cue. As one example, the feedback device 18 can include a green light, a yellow light, and a red light, where the green light is displayed when the measured parameter is below the threshold value for that parameter by a predetermined amount, the yellow light is displayed when the measured parameter is approaching the threshold value, and the red light is displayed when the measured parameter extends beyond the threshold value. Other exemplary ways in which the feedback device 18 can alert the individual 12 include generating an audible alarm and/or vibrating. Embodiments in which the feedback device 18 alerts the individual 12 with a haptic cue (e.g., a vibration) can be employed to maintain the individual's privacy, while still informing the individual 12 that the threshold value has been exceeded. The magnitude and/or duration of the alarm or vibration can relate proportionally or otherwise to the magnitude of the measured value, as compared to the threshold value or range.

In various embodiments, the threshold values can be periodically altered such that the individual 12 can experience additional levels of activity without being alerted by the feedback device 18. In some cases, this approach can enable a progressive return to full activity for an individual recovering from an injury. Taking an example of the system 10 being used to monitor a patient recovering from mTBI, one parameter the sensor 14 may measure is the individual's heart rate. Research indicates that individuals with mTBI should restrict their heart rate to a low level in the initial stages of recovery (although complete physical rest is not advised, as it can have negative consequences), and gradually increase their heart rate in a tiered fashion until they return to full activity. The system 10 can assist an individual 12 in implementing this recovery approach. For example, the threshold value for heart rate can initially be set at 80 beats per minute (“bpm”), such that the individual 12 will be alerted by the feedback device 18 if his or her heart rate exceeds this amount. Alternatively, in embodiments in which the feedback device includes green, yellow, and red lights, the green light may be on when heart rate is below 70 bpm, the yellow light may be on when heart rate is between 70 and 80 bpm, and the red light may be on when heart rate exceeds 80 bpm. At the same time, the individual 12 can be assured that participating in activities that do not result in an alert from the feedback device 18 are appropriate. Thus, the system 10 can enable the individual 12 to keep his or her heart rate in a desired range during recovery by authorizing activity levels that do not result in an alert from the feedback device 18 (thereby avoiding the negative consequences associated with complete physical rest), but also alerting the individual 12 if his or her heart rate reaches a level that can impede the recovery process.

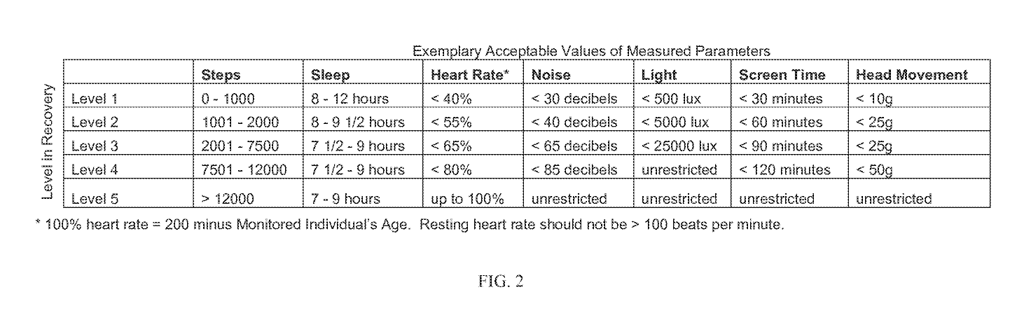

At a predetermined or programmable interval, the heart rate threshold value can be increased such that the individual's heart rate can reach increased levels without the individual 12 receiving an alert from the feedback device 18 (e.g., mobile device 26). In general, the threshold values can be altered on any desired schedule, for example every day, week, month, or on an irregular schedule. In some cases, the controller 16 can automatically alter the threshold values at set intervals. In other cases, the threshold values are altered upon the controller 16 receiving instruction from the individual 12 or a third-party (e.g., through engaging the interface 20 defined below). In some instances, the controller 16 can be pre-programmed with a threshold value for each measured parameter at each interval and/or with an operative function for altering the threshold value at each interval (e.g., increase the threshold value by 10% every three days). In other instances, the individual 12 or a third-party (e.g., a nurse or physician) can program the controller 16 with the threshold values. For the purpose of providing a non-limiting illustration of the concept described above, in some embodiments the threshold value for heart rate can be altered such that at the end of each 24 hour period, the threshold value is increased by 10 bpm. Taking the example given above, after 24 hours the individual may only be alerted if heart rates exceeds 90 bpm, after 48 hours the individual may only be alerted if heart rate exceeds 100 bpm, etc. Although the above description focuses on the example parameter of heart rate, any parameter monitored by the system 10 can have its threshold value altered in a similar fashion. Various parameters can be altered similarly or at different times and in different increments as desired. FIG. 2 is a chart showing some exemplary acceptable values of measured parameters at various levels. As shown, the measured parameters may include: steps taken, amount of sleep, heart rate, noise exposure, light exposure, smart device screen exposure, and/or head movement. The head movement parameter is expressed in g-force units. Measurement of this parameter may be used to ensure that a recovering patient does not experience excessive g-force from relatively short impact events that may occur during physical (e.g., athletic) activity. For example, a standard football tackle may impart a 40 g-110 g force, but for a relatively short period of time (e.g., only during the impact of the tackle). The measured parameter shown in the chart may allow a recovering patient to periodically work back to being able to handle this type of force. Of course, human capacity for g-force over longer durations is much lower; for example, some sources indicate that experiencing a 16 g-force for a minute can be deadly. This experience of g-force over a longer duration may be a different measured parameter, not shown in FIG. 2. In general, the measured parameters and acceptable values are shown for purposes of illustrating the concept of periodically altering acceptable values as described herein and are non-limiting of the invention. Other parameters may be measured, and other acceptable values may be used.

In certain embodiments, the system 10 can include an interface 20 adapted to be engaged by the individual 12 or a third party. In general, the interface 20 can be located on any part of the system 10, for example, the sensor 14, the feedback device 18, or in some cases as a stand-alone device. As described above, in some instances the individual 12 can engage the interface 20 to instruct the controller 16 to alter the threshold values. In some instances, the individual 12 can engage the interface 20 to signal to the system 10 that the individual 12 is experiencing discomfort, or in some cases, not experiencing discomfort. In such cases, in general, the controller 16 can alter the parameter threshold values in response to such communication (e.g., if the user indicates discomfort, to alleviate such discomfort). For example, the controller 16 can reduce the threshold values to a previous level. Taking the example alteration schedule of the heart rate parameter given above, if the individual 12 engages the interface 20 signaling discomfort during the time in the recovery process when the heart rate threshold value is 90 bpm, the controller 16 can reduce the heart rate threshold value back to 80 bpm. After another interval, the controller can resume gradually altering the threshold value until the individual 12 returns to full-activity levels. In providing this capability, the system 10 can ensure that monitored individuals are asymptomatic (or symptoms are acceptable) before moving on to increased levels of activity. In other instances, if the user signals no discomfort, the controller can alter the threshold levels accordingly (e.g., on a more accelerated schedule).

Although the disclosure has primarily provided examples regarding the system's monitoring of the individual's heart rate, as mentioned, the system 10 can measure numerous parameters, examples of which are provided in the following description. In an embodiment in which the system 10 is used to assist the individual 12 in recovery from mTBI, the system 10 can measure and provide feedback on three broad categories of parameters: (1) physical activity, (2) cognitive activity, and (3) sleep.

Within the physical activity category, the system 10 can measure and provide feedback to the individual 12 regarding heart rate as described above. In addition, the heart rate data may be used to calculate heart rate variability (e.g., using an algorithm logic generated from the measured heart rate data). In a similar fashion, the system 10 can measure and provide feedback regarding the individual's body temperature. Also within the physical activity category, the system 10 can measure and provide feedback regarding the motion of certain parts of the individual's body. For an individual recovering from mTBI, it can be useful to monitor motion of the individual's head. Minor head movements caused by seemingly innocuous behavior, like riding a bus to school, sexual activity, or low level physical activity, has been linked to delayed recovery and additional brain injury for those diagnosed with mTBI.

Accordingly, the sensor 14 can measure the linear, rotational, and/or angular acceleration of the individual's head and provide feedback when such acceleration exceeds an acceptable threshold value. In order to record such measurements, the sensor 14 may include a multi-axis accelerometer.

The cognitive activity category can include direct measurements of the individual's cognitive activity. For example, the sensor 14 can measure an individual's cognitive exertion, for example, using an electroencephalography (EEG) sensor, and the feedback device 18 can alert the individual when such exertion extend beyond a threshold value.

Within the cognitive activity category, the system 10 may provide a reminder to the individual 12 to take periodic breaks from cognitive activity during non-sleeping hours.

Research has indicated that frequent cognitive breaks (e.g., periods during which the individual 12 is not taxing their brain) can help the individual 12 recover from mTBI. Accordingly, the feedback device 18 (e.g., mobile device 26) may provide a periodic reminder (e.g., with a visual, audible, and/or haptic cue) to the individual 12 to take such breaks. As with other recovery parameters, cognitive breaks may be implemented in a progressive manner such that the frequency of the cognitive break reminders may decrease as the individual's recovery progresses.

The cognitive activity category can also include parameters related to the individual's sensory exposure. An individual's environment is sensory rich, which can make it difficult for the brain to rest when experiencing mTBI. Sensitivity to light and noise are common amongst individuals diagnosed with mTBI. The adverse effects of noise, including increased reports of fatigue, headache, and irritability, on both learning and work environments is well documented. Accordingly, the system 10 can measure and provide feedback related to the individual's sensory exposure. For example, the sensor 14 can measure the individual's light level exposure (e.g., lux levels and/or RGB levels), which can include ambient light, and the feedback device 18 can alert the individual 12 when such levels exceed a threshold value. The sensor 14 can also measure the individual's noise level exposure, and the feedback device 18 can alert the individual when such levels exceed a threshold value. In some cases, the mobile device 26 can be the sensor 14 and/or the feedback device 18 measuring and/or providing feedback on the individual's exposure to light and noise.

The cognitive activity category can also include the individual's use of a mobile device 26, for example, a smartphone or tablet computing device. Studies have shown that Americans check their smartphone an average of 150 times, and spend an average of 2 hours and 38 minutes on their smartphone or tablet, each day. In some situations, use of such devices can be cognitively taxing and impede recovery from conditions such as mTBI. Accordingly, the system 10 can measure and provide feedback to the individual 12 regarding mobile device usage. In some instances, the sensor 14 can be adapted to measure the amount of usage (e.g., time and/or data consumption) of the individual 12 for a particular mobile device 26, and the feedback device 18 can alert the individual 12 when such usage exceeds a threshold value. In other embodiments, the system 10 can include an application located on and executed by the mobile device 26 that measures the mobile device usage. In such embodiments, the mobile device 26 may function as the feedback device 18, as well. For example, the individual 12 may receive visual alerts within the mobile device's user interface indicating that the usage threshold value has been exceeded.

Within the sleep category, the system 10 can measure and provide feedback to the individual 12 regarding the amount of sleep received by the individual 12. Amount of sleep can be an important parameter in recovery from mTBI, as lack of sleep has been shown to produce adverse effects, such as headaches and irritability, in individual's diagnosed with mTBI. Many individuals are unaware that difficulty with sleep is even a problem associated with mTBI and/or rely on subjective measurements of sleep. Accordingly, the system 10 can measure the amount of sleep received by the individual 12. In some instances, sleep can be measured using an algorithm logic generated from an accelerometer. In such instances, the sensor 14 may include an accelerometer which can be located on any part of the individual's body, for example, the individual's wrist or head. The algorithm logic may differentiate between intervals in which the individual 12 is awake and asleep. This type of measurement is sometimes referred to as actigraphy. In another instance, sleep can be measured using an algorithm logic generated from an accelerometer and a heart rate measurement. This type of measurement is sometimes referred to as ballistocardiography. The feedback device 18 (e.g., mobile device 26) can alert the individual 12 if the amount of sleep received falls below (or exceeds) a threshold value. The sleep category of parameters can also include the individual's sleep environment. Exposure to light, for example the blue light that is emitted by smartphones and tablets, may have a negative effect on sleep quality and therefore directly affect recovery from certain conditions such as mTBI. Ensuring a room is dark and free of noise can be a first step to ensuring the individual 12 receives proper sleep. Accordingly, the system's measurement and provision of feedback regarding the individual's sensory environment described above, can include measuring and providing feedback regarding the light and noise levels in the individual's sleep environment.

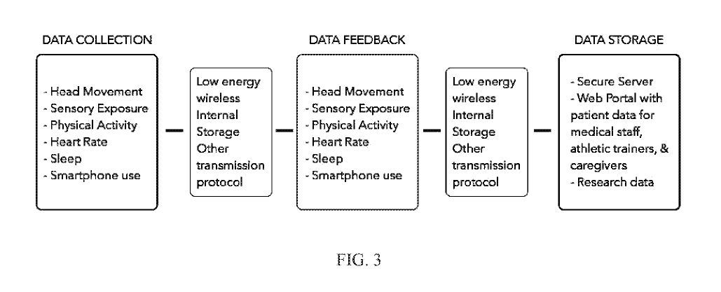

In various embodiments, the system 10 can locally store the data it collects in a memory 22, and in some instances can transfer the data to a remote storage device 24. The data collected by the system 10 can include, for example, the measurements taken by the sensor 14, as well as data regarding the feedback device's alerts to the individual (e.g., frequency of alerts, time stamps of alerts, and the individual's response to alerts). In some cases, the remote storage device 24 can also receive data collected by the individual's mobile device 26, such as usage data. The remote storage device 24 can allow the monitored individual 12 and/or a third party (e.g., a physician or a caregiver) to access the data. For example, the remote storage device 24 may be a server that hosts the communicated data on a web portal. In some cases, such a system may also enable a third party to alter the threshold levels (e.g., remotely) based on the measured data. As one example, if a physician interprets the measured data to indicate that a patient is recovering faster than typical, the physician may remotely alter the threshold levels appropriately. In some instances, the remote storage device 24 may aggregate the collected data from numerous individuals and make it available (e.g., anonymously, with all personally identifiable information removed) such that research can be conducted on the various conditions that can be monitored by the system 10. FIG. 3 is a flow diagram showing example transfers of collected data. FIG. 3 only illustrates limited examples of collected data and data transfer methods; other types of data may be collected and other data transfer techniques may be used.

Although the above description focuses primarily on the system 10 being used in assisting the individual 12 in recovering from a diagnosed condition, particularly mTBI, in certain embodiments the system 10 can be used in any situation in which a parameter is measured, feedback is provided when the parameter exceeds (or in some cases, falls below) a threshold value, and the threshold value is periodically altered. One example of such a situation can include an athlete following an athletic training regimen. In such a situation, the training regimen may have certain measurable parameters, for example, distance the athlete runs, amount the athlete weighs, and the athlete's heart rate. In a similar fashion as described above for assisting an individual recover from a condition, the system 10 can measure such parameters, provide feedback to the athlete when such parameters exceed a threshold value, and periodically alter the parameters throughout the training regimen. Another example of such a situation can include an individual following a regimen for a mental health condition. Anxiety, depression, and posttraumatic stress disorder (“PTSD”) are all commonly diagnosed conditions that affect millions of individuals. Once diagnosed, patients are often directed to change their behavior in order to reduce causing, provoking, or worsening symptoms. As an example, individuals diagnosed with anxiety and PTSD can benefit from exposing themselves to increasing levels of sensory exposure. In a similar fashion as described above for assisting an individual recover from a condition, the system 10 can measure relevant sensory exposure parameters, provide feedback to the patient when such parameters exceed a threshold value, and periodically alter the parameters throughout the regimen.

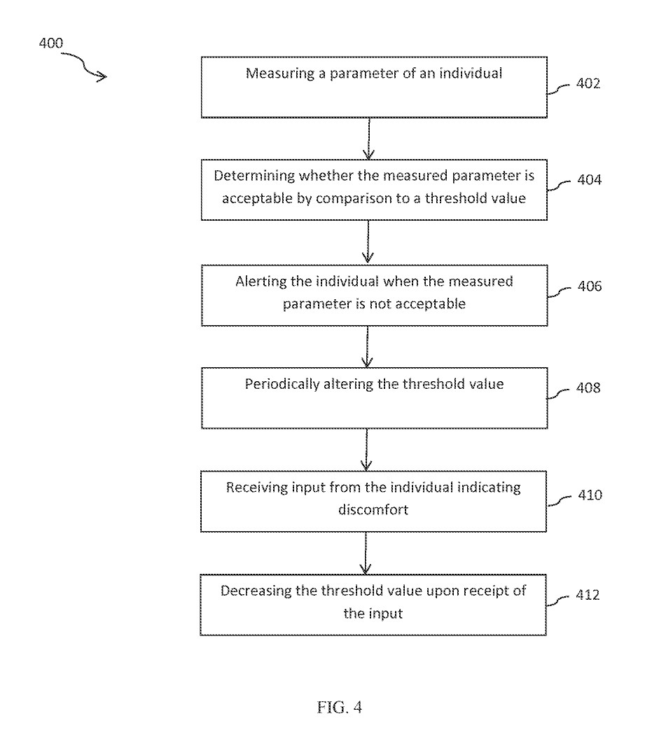

FIG. 4 is a flow diagram showing an example method 400 for monitoring an individual in accordance with an embodiment of the invention. The method 400 may include the step 402 of measuring a parameter of an individual. For example, the parameter can relate to the individual's movement, physiological function, and/or environment. The method 400 may include the step 404 of determining whether the measured parameter is acceptable by comparison to a threshold value or range of values, and the step 406 of alerting the individual when the measured parameter is not acceptable. The alerting step 406 can include providing the individual with a visual, audible, and/or haptic cue. The method 400 can include the step 408 of periodically altering the threshold value, which can include increasing or decreasing the threshold value. In some embodiments, the method 400 can include the step 410 of receiving input from the individual indicating the individual's discomfort with a current situation or experience, and in some cases can include the step 412 of decreasing (or increasing) the threshold value upon receipt of the input.

This input step 410 can be very effective in tailoring the recovery or improvement to the individual. The input step 410 can be signaled with a special button (e.g., a blue button) on the interface 20, the controller 16, the feedback device 18, the mobile device 26 or via any other appropriate input scheme. Empowering the individual 12 with control over the recovery schedule can be a very effective function. This helps to ensure user adoption and compliance with the recovery scheme, without subjecting the individual to a particular regimen or schedule of activity and exposure.

The terms and expressions employed herein are used as terms and expressions of description and not of limitation, and there is no intention, in the use of such terms and expressions, of excluding any equivalents of the features shown and described or portions thereof In addition, having described certain embodiments of the invention, it will be apparent to those of ordinary skill in the art that other embodiments incorporating the concepts disclosed herein may be used without departing from the spirit and scope of the invention. The features and functions of the various embodiments may be arranged in various combinations and permutations, and all are considered to be within the scope of the disclosed invention. Accordingly, the described embodiments are to be considered in all respects as only illustrative and not restrictive. Furthermore, the configurations described herein are intended as illustrative and in no way limiting. Similarly, although physical explanations have been provided for explanatory purposes, there is no intent to be bound by any particular theory or mechanism, or to limit the claims in accordance therewith.

What is claimed is:

1. A wearable, sensor-based system for monitoring an individual, the system comprising:
a sensor adapted to measure a parameter related to at least one of movement, a physiological function, and environment of the individual;
a controller adapted to receive sensor measurement and determine whether the sensor measurement is acceptable by comparison to a threshold value; and
a feedback device in communication with the controller adapted to alert the individual when the sensor measurement is not acceptable, wherein the controller is further adapted to periodically increase the threshold value.

2. The system of claim 1, wherein the sensor comprises a plurality of different parameter measurement elements.

3. The system of claim 1, wherein the sensor comprises a headband adapted to be worn by the individual.

4. The system of claim 1, wherein the movement parameter is selected from the group consisting of linear acceleration, rotational acceleration, and angular acceleration of a head of the individual and combinations thereof.

5. The system of claim 1, wherein the physiological function parameter is selected from the group consisting of heart rate, body temperature, cognitive activity, and amount of sleep.

6. The system of claim 1, wherein the environment parameter is selected from the group consisting of ambient light level and ambient noise level.

7. The system of claim 1, wherein the feedback device comprises a wearable device separate from the sensor.

8. The system of claim 7, wherein the feedback device comprises a wristband.

9. The system of claim 1, wherein the feedback device alerts the individual with at least one of a visual cue, an audible cue, and a haptic cue.

10. The system of claim 1, wherein at least one of the feedback device and the sensor communicate wirelessly with the controller.

11. The system of claim 1, wherein the controller is further adapted to receive input from the individual indicating at least one of presence and absence of discomfort.

12. The system of claim 11, wherein the feedback device comprises an interface adapted to be engaged by the individual to indicate at least one of presence and absence of discomfort and to notify the controller.

13. The system of claim 11, wherein the controller is further adapted to decrease the threshold value upon receiving a communication indicating discomfort.

14. The system of claim 13, wherein the threshold value is decreased to a previous level.

15. The system of claim 1, wherein the feedback device comprises a mobile telecommunications device.

16. The system of claim 15, wherein the mobile telecommunications device comprises at least one of a smartphone and a smartwatch.

17. The system of claim 15, wherein the parameter further relates to mobile telecommunication device usage.

18. The system of claim 1, wherein the controller is further adapted to communicate data to a remote data storage device.

19. The system of claim 18, wherein the remote storage device comprises a server that hosts the communicated data on a web portal.

20. A method of using a wearable, sensor-based device for monitoring an individual, the method comprising the steps of:
measuring a parameter related to at least one of movement, a physiological function, and environment of the individual;
determining whether the measured parameter is acceptable by comparison to a threshold value;
alerting the individual when the measured parameter is not acceptable; and
periodically increasing the threshold value.

21-27. (canceled)
